(19) United States
(12) Patent Application Publication
(10) Pub. No.: US 2025/0127612 A1
(43) Pub. Date: Apr. 24, 2025

(54) PERCUTANEOUS TRICUSPID VALVE REPLACEMENT DEVICES AND METHODS

(71) Applicant: Tangent Cardiovascular, Inc., Santa Rosa, CA (US)

(72) Inventor: Nathan Daniel Brown, Forestville, CA (US)

(21) Appl. No.: 18/918,615

(22) Filed: Oct. 17, 2024

Related U.S. Application Data
(60) Provisional application No. 63/592,705, filed on Oct. 24, 2023.

(51) Int. Cl. A61F 2/24

(52) U.S. Cl. CPC A61F 2/2412; A61F 2220/0008

(57) ABSTRACT

A prosthetic heart valve is provided. The prosthetic heart valve includes a central body portion and a tether-capture arm that extends from the central body portion. The tether capture arm forms a U-shaped tether-capture notch captures and retains a tether that spans the native heart valve annulus.

### CROSS-REFERENCES TO RELATED APPLICATIONS

This application claims priority to U.S. Provisional Application No. 63/592,705, filed Oct. 24, 2023, and entitled “PERCUTANEOUS TRICUSPID VALVE REPLACEMENT DEVICES AND METHODS,” the disclosure of which is hereby incorporated by reference in its entirety. The present application further hereby incorporates by reference in its entirety the subject matter of International PCT Patent Application No. PCT/US2023/020277 entitled “PERCUTANEOUS TRICUSPID VALVE REPAIR DEVICES AND METHODS,” filed Apr. 27, 2023.

### BACKGROUND

### Field

The present disclosure generally relates to percutaneous replacement of a heart valve and in certain embodiments percutaneous replacement of a tricuspid valve.

### Description of the Related Art

Functional tricuspid regurgitation (FTR) is a result of one or several pathophysiological abnormalities of the heart which may include tricuspid valve annular dilatation, annular shape, right or left ventricular disfunction, right or left ventricular shape, right or left sided heart failure, pulmonary hypertension (primary or secondary to mitral valve regurgitation), and valve leaflet tethering. It is currently estimated that 1.6 million people in the United States suffer from moderate to severe tricuspid regurgitation and only 8,000 of these people undergo tricuspid valve surgery annually. Historical treatment options for patients suffering from tricuspid regurgitation have been medical management and tricuspid valve surgery. Most tricuspid valve surgeries are in conjunction with left heart valve surgery, for instance, mitral valve surgery. Few isolated tricuspid valve surgeries are performed and those that are performed are associated with high operative mortality (8-10%). FTR, particularly moderate to severe FTR, has been shown to be a strong independent determinant of mortality and is associated with more heart failure signs and symptoms, reduced cardiac output and impaired renal function. Thus, there is a distinct need for less invasive approaches to treating FTR.

### SUMMARY

Certain embodiments of the disclosure comprise implanting a prosthetic replacement heart valve into the tricuspid annulus, such prosthetic heart valve being configured to integrate with a percutaneous tricuspid repair system such as the embodiments of tricuspid valve repair systems found in International PCT Patent Application No. PCT/US2023/020277 entitled “PERCUTANEOUS TRICUSPID VALVE REPAIR DEVICES AND METHODS,” filed Apr. 27, 2023, the entire contents of which are incorporated herein by reference. In some aspects, the prosthetic heart valve can be configured to displace the leaflets of the native tricuspid valve radially and replace the function of the native tricuspid valve for the purpose of regulating blood flow between the right atrium and right ventricle of a heart. In certain arrangements the prosthetic valve is configured with one or more tether capture arms attached to the superior portion of the valve frame. The tether capture arms can radiate inward from the perimeter of the valve frame prior to forming an arc that radiates the tether capture arms back toward the perimeter, thereby forming an arc of a partial circle such that the tether capture arms can wrap around tethers that span the tricuspid annulus as described in PCT/US2023/020277. In certain arrangements, the prosthetic heart valve can include a right ventricular outflow tract (RVOT) capture arm that extends from the central body portion of the prosthetic heart valve (e.g., the prosthetic heart valve frame). The RVOT-capture arm can be flexible and can traverse inferior to the native tricuspid valve annulus and into the RVOT. The RVOT-capture arm can be sized to exert pressure on the inner surface of the RVOT and contribute to the fixation of the prosthetic heart valve within the native valve annulus. In certain variations, the prosthetic heart valve can be configured with a covering, which can include an inflow flange on the superior (inflow) surface of the prosthetic heart valve. The inflow flange and the covering layer of the central body portion of the prosthetic heart valve can be configured to prevent or reduce blood from flowing freely through the perimeter of the prosthetic heart valve. In some aspects, the covering layer and the inflow flange can contribute to sealing against the native tricuspid valve leaflets and annulus. In some aspects, the inflow flange can be configured to lie against the right atrial surface of the native tricuspid valve annulus and contribute to sealing. In some arrangements, the inflow flange can contribute to fixation of the prosthetic heart valve against the native tricuspid valve annulus to reduce or prevent migration of the prosthetic heart valve. In certain embodiments the prosthetic heart valve can include a posterior support arm that radiates from the inferior end of the central body portion of the prosthetic heart valve. The posterior support arm can be disposed approximately opposite circumferentially to the RVOT-capture arm and can be configured to brace against the posterior tricuspid valve annulus to reduce or mitigate rotation of the prosthetic heart valve under hemodynamic forces. In some arrangements, the covering layer can extend over one or both or neither of the RVOT-capture arm and posterior support arm. In certain aspects, the prosthetic heart valve can include a compliant central body portion designed to be implanted longitudinally between the tethers of a tricuspid valve repair system such that the central body portion displaces the tethers radially while conforming around the tethers such that the tethers create an indentation or tether-capture notch in the perimeter of the central body portion such that the tethers secure the prosthetic heart valve in place. In some aspects, the prosthetic heart valve replacement system disclosed herein can be composed of a tricuspid valve repair system having one or more tethers that span the native tricuspid valve annulus and a prosthetic heart valve with a flange on the inflow side, such flange positioned between the tethers and the tricuspid valve annulus such that the prosthetic heart valve is retained between the tethers and the tricuspid valve annulus.

In some aspects, the techniques and devices described herein relate to a tricuspid valve replacement system including one or more features of the foregoing description.

In some aspects, the techniques described herein relate to a method of tricuspid valve replacement including implanting one more features of the tricuspid valve replacement system of the foregoing description into a patient's heart.

### BRIEF DESCRIPTION OF THE DRAWINGS

These and other features, aspects, and advantages of the present application are described with reference to drawings of certain embodiments, which are intended to illustrate, but not to limit, the present inventions. It is to be understood that these drawings are for the purpose of illustrating the various concepts disclosed herein and may not be to scale.

FIG. 1 illustrates a prosthetic heart valve implanted in a tricuspid annulus between a right atrium and a right ventricle, according to some aspects of the present disclosure.

FIG. 2 illustrates a right-atrium view of a prosthetic heart valve implanted in a tricuspid annulus between a right atrium to a right ventricle, according to some aspects of the present disclosure.

FIG. 3 illustrates a partial side and superior view of a prosthetic heart valve, according to some aspects of the present disclosure.

FIG. 4 illustrates a partial side and superior view of a prosthetic heart valve, according to some aspects of the present disclosure.

FIG. 5 illustrates a partial side and superior view of a prosthetic heart valve, according to some aspects of the present disclosure.

FIG. 6 illustrates a side view of the prosthetic heart valve of FIG. 5.

FIG. 7 illustrates a top view of the prosthetic heart valve of FIG. 5.

FIG. 8 illustrates a side view of the prosthetic heart valve of FIG. 4.

FIG. 9 illustrates a side view of the prosthetic heart valve, according to some aspects of the present disclosure.

FIG. 10 illustrates a top view of the prosthetic heart valve of FIG. 4.

FIG. 11 illustrates a top view of the prosthetic heart valve, according to some aspects of the present disclosure.

FIG. 12 a prosthetic heart valve implanted in a tricuspid annulus between a right atrium and a right ventricle, according to some aspects of the present disclosure.

FIG. 13 illustrates a side view of the prosthetic heart valve of FIG. 12.

FIG. 14 illustrates a side view of a prosthetic heart valve, according to some aspects of the present disclosure.

FIG. 15 illustrates a long-axis cross-sectional view of a prosthetic heart valve implanted in a tricuspid annulus between a right atrium and a right ventricle, according to some aspects of the present disclosure.

FIG. 16 illustrates a prosthetic heart valve implanted in a tricuspid valve annulus between a right atrium and a right ventricle, according to some aspects of the present disclosure.

FIG. 17 illustrates a method of implanting a prosthetic heart valve in a tricuspid valve annulus between a right atrium and a right ventricle, according to some aspects of the present disclosure.

### DETAILED DESCRIPTION OF EMBODIMENTS

Embodiments of the disclosure will now be described with reference to the accompanying figures. The terminology used in the description presented herein is not intended to be interpreted in any limited or restrictive manner, simply because it is being utilized in conjunction with a detailed description of certain embodiments of the disclosure. Furthermore, embodiments of the disclosure may include several novel features, no single one of which is solely responsible for its desirable attributes or which is essential to practicing the embodiments of the disclosure herein described. For purposes of this disclosure, certain aspects, advantages, and novel features of various embodiments are described herein. It is to be understood that not necessarily all such advantages may be achieved in accordance with any particular embodiment. Thus, for example, those skilled in the art will recognize that one embodiment may be carried out in a manner that achieves one advantage or group of advantages as taught herein without necessarily achieving other advantages as may be taught or suggested herein.

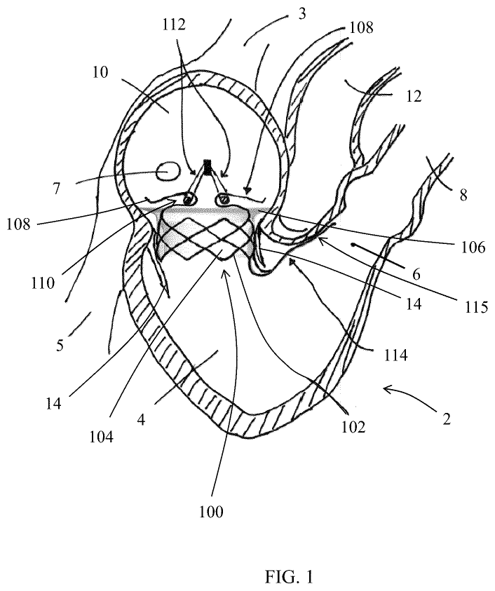

FIG. 1 illustrates a cross section of a heart 2 showing a long axis view of the right ventricle 4, the RVOT 6, the pulmonary artery 8 and the right atrium 10. FIG. 1 also shows the superior vena cava 3, the inferior vena cava 5, and the coronary sinus 7, which each supply blood to fill the right atrium 10. The left ventricle is not shown in FIG. 1, but the aorta 12, which receives the blood ejected from the left ventricle, is shown in partial cross section. An illustrative, non-limiting arrangement of a prosthetic heart valve 100 is shown in FIG. 1, according to some aspects of the present disclosure, and the prosthetic heart valve 100 is depicted seated within the native tricuspid valve annulus that connects the right atrium 10 to the right ventricle 4. In some aspects, the native tricuspid valve leaflets 14 can be displaced radially outward by the presence of the prosthetic heart valve 100, as shown in FIG. 1. In some arrangements, the native tricuspid valve leaflets 14 can be removed (e.g., resected) partially or completely during implantation of the prosthetic heart valve 100.

The prosthetic heart valve 100 can have a central body portion 102 that provides structural support to the prosthetic heart valve 100, as described herein. In some aspects, the central body portion 102 can be sized to define a flow path for blood to pass through the prosthetic heart valve 100 (e.g., from the right atrium 10 into the right ventricle 4). The central body portion 102 can define a generally hollow cylindrical shape, or tube-like structure, and can be movable (e.g., expanded) from a small-diameter-delivery configuration to a large-diameter-deployed configuration, as described herein. The prosthetic heart valve 100 can include a covering layer 104 disposed over the central body portion 102. The covering layer 104 can be configured to prevent blood from flowing radially through the central body portion 102. In some arrangements, the prosthetic heart valve 100 can include an inflow flange 106 at the atrial or superior end of the prosthetic heart valve 100, as shown in FIG. 1. The inflow flange 106 can extend radially beyond the outer perimeter of the central body 102 such that an outer dimension of the inflow flange 106 is larger than (e.g., disposed radially outward from) an outer dimension of the superior end of the central body 102. The inflow flange 106 can be configured to seal against the atrial side of the tricuspid valve annulus to prevent perivalvular blood flow between the prosthetic valve 100 and the native heart tissue. In other words, the inflow flange 106 can be configured to ensure blood flow from the right atrium 10 to the right ventricle 4 passes through the flow channel defined by the central body portion 102.

In some aspects, the prosthetic heart valve 100 can include one or more tether-capture arms 108 configured to prevent or reduce migration of the prosthetic heart valve 100, as described herein. In the non-limiting, illustrative arrangement shown in FIG. 1, the prosthetic heart valve 100 has two tether-capture arms 108 that are diametrically opposed to one another with each tether-capture arm 108 extending from the superior or inflow side of the central body portion 102. The tether-capture arms 108 have a U-shaped geometry that folds back onto itself to create a tether notch 110 configured to capture and retain within the tether notch 110 a tether 112 that extends from the heart wall, as described herein. In FIG. 1, two tethers 112 are shown traversing from an interatrial septal location into the illustration. Such tethers 112 can comprise, for example, components of a tricuspid valve repair system such as the embodiments of tricuspid valve repair systems described in PCT/US2023/020277 and referred to herein as the tricuspid valve repair device or system and tricuspid valve repair device or system tethers.

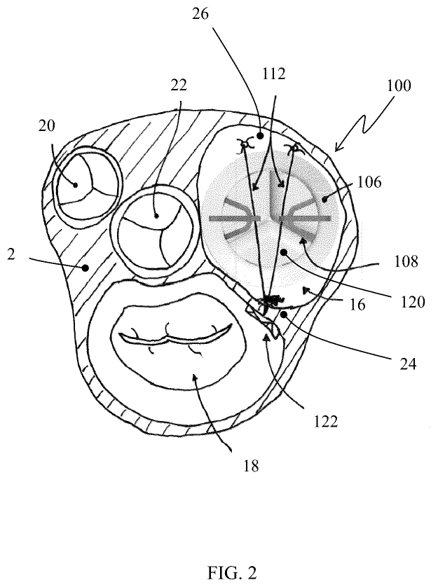

With continued reference to FIG. 1, the prosthetic heart valve 100 is illustrated in the native tricuspid valve location, with the prosthetic heart valve 100 displacing the native tricuspid valve leaflets 14 and positioned within the native tricuspid valve annulus 16 (shown in FIG. 2). The prosthetic heart valve 100 is illustrated in FIG. 1 with a central body portion 102 having a perimeter that houses the prosthetic valve leaflets 120 (FIG. 2), as described herein. The prosthetic heart valve 100 shown in FIG. 1 has a pair of tether-capture arms 108 on the superior (inflow) end of the central body portion 102 and has a RVOT-capture arm 114 on the inferior (outflow) end of the central body portion 102. The RVOT-capture arm 114 can be sized to extend into the RVOT 6 to help stabilize the prosthetic heart valve 100, as described herein. The prosthetic heart valve 100 illustrated in FIG. 1 is depicted to further include an inflow flange 106 that joins to the covering layer 104 at the inflow or superior perimeter of the central body portion 102. The inflow flange 106 can be sized to contact the native tissues to prevent free blood flow through the perimeter of the prosthetic heart valve 100. The covering layer 104 can be connected to, or monolithic with, the inflow flange 106, which rests against the right atrial surface of the native tricuspid valve annulus. The covering layer 104 further prevents or reduces free blood flow through the perimeter the prosthetic heart valve 100 and prevents or reduces free blood flow through the central body portion 102. The covering layer 104 can also provide a frictional contact surface that presses against surrounding native heart tissue (e.g., native valve annulus, native valve leaflets) to reduce or eliminate migration of the prosthetic heart valve 100.

The structural frame of the central body portion 102 is preferably constructed as an expanding metallic form such that the prosthetic heart valve 100 can be crimped, squeezed, or otherwise compressed into a first state (e.g. a compressed or delivery configuration) where the diameter of the prosthetic heart valve 100, or the inner diameter of a tube that could enclose the prosthetic heart valve 100, is substantially smaller than a second state (e.g., a deployed or expanded configuration) where the prosthetic heart valve 100 is fully expanded and the prosthetic heart valve 100 is competent (e.g., reduces or eliminates FTR). The prosthetic heart valve 100 can therefore be constrained in the first state for the purpose of inserting the prosthetic heart valve 100 into a catheter for minimally invasive guidance into a patient's heart, and upon release of the constraint, the prosthetic heart valve 100 can resume the second state whereby the prosthetic heart valve 100 is expanded to a diameter suitable for regulating blood flow between the right atrium and right ventricle and for sealing with the native structures of the tricuspid valve. For example, the first state can be a diameter between 4 mm and 13 mm, which would be suitable for insertion and tracking through a patient's femoral vein, and the second state can be a diameter between 25 mm and 60 mm, which would be suitable for replacing a patient's native tricuspid valve.

Expansion of the prosthetic heart valve 100 between the first and second state can be achieved through self-expansion, where the stored strain energy from compressing the prosthetic heart valve 100 is released upon removal of a restraint, such as a catheter. Alternatively, expansion can be achieved by mechanical means, for example, by expanding the prosthetic heart valve 100 with a balloon inflated from within the inner diameter of the central body portion 102. In the case where the prosthetic heart valve 100 is self-expanding, the prosthetic heart valve 100 can be constructed of a super-elastic material, such as nitinol, and in the case where the prosthetic heart valve 100 requires mechanical expansion, the prosthetic heart valve 100 can be constructed from metals suitable for long term implantation in a patient's heart such as cobalt chromium or stainless steel. As shown in FIG. 1, the prosthetic heart valve 100 can further include a central body portion 102 configured as a series or a web of thin struts that form a diamond pattern around the perimeter of the central body portion 102 such that the perimeter has some compliance and such that large radial displacements of the central body portion 102 exert minimal (less than 20%) strain in the material.

The tether-capture arms 108 are preferably flexible such that that they can be elongated and compressed to facilitate insertion into a delivery catheter while maintaining enough stiffness to support the prosthetic heart valve against the hemodynamic forces across the valve orifice. In certain arrangements, the tether-capture arms 108 can begin at the perimeter of the central body portion 102 on the superior side and can be transposed inward toward the center of the central body portion 102 prior to forming an arc and then transposing back toward the perimeter. The partial loop created by the arced path of the tether-capture arms 108 makes it possible to encircle and capture the tethers 112 (e.g., tethers of a tricuspid valve repair device). By capturing the tethers 112 of a tricuspid valve repair device, the tether-capture arms 108 can utilize the tricuspid valve repair device tethers 112 to increase fixation of the prosthetic heart valve 100 within the native tricuspid valve annulus. Thus, the tricuspid valve repair device is incorporated into the prosthetic tricuspid valve replacement and provides resistance to migration in the inferior and superior directions. Significantly, this reduces the need to rely on alternative fixation methods, such as radial force, barbs, hooks, or elements that could interact with the native chordae tendinea.

By reducing reliance on radial force, the prosthetic heart valve 100 can minimize pressure on the atrio-ventricular node (AV node), which can result in less conduction disturbances compared to alternative tricuspid valve replacement devices described in the art. In certain variants, the tether-capture arms 108 can be configured to exert a force on the tethers 112 directed toward the perimeter of the prosthetic heart valve 100, such force effectively shortening the tethers 112 along their primary axis, which can effectively draw the right ventricular free wall toward the interatrial septum and improve right heart function. The tether-capture arms 108 are preferably constructed from the same material as the central body portion 102 of the prosthetic heart valve 100 without the use of joints or connections. Suitable methods of construction can include laser cutting from a solid tube or braiding or weaving a wire or wires.

The RVOT-capture arm 114 can be preferably flexible such that the RVOT-capture arm 114 can be elongated (e.g., brought into a low-profile configuration for delivery and implantation of the prosthetic heart valve 100) and compressed to facilitate insertion of the prosthetic heart valve 100 into a delivery catheter while maintaining enough stiffness to support the prosthetic heart valve 100 against the hemodynamic forces across the valve orifice once the RVOT-capture arm 114 and the prosthetic heart valve 100 are expanded into the fully-competent-valve configuration. The RVOT-capture arm 114 can be preferably configured to extend radially outward from the inferior or downstream end of the central body portion 102 and into the RVOT 6. In some aspects, the RVOT-capture arm 114 can be further configured such that in a free state (e.g., the expanded configuration) the distal portion 115 of the RVOT-capture arm 114 curls back toward the central body portion 102. However, when implanted in the tricuspid valve of a heart 2, the RVOT-capture arm 114 will contact the superior surface of the RVOT 6 (as shown in FIG. 1) and exert a force that pulls the prosthetic heart valve 100 inferiorly. This inferiorly directed force is preferably counteracted by a superiorly directed force between the tether capture arms 108 and the tethers 112, and, in some arrangements, between the inflow flange 106 and the right atrial surface of the tricuspid annulus, thus fixating and stabilizing the prosthetic heart valve 100 between the tether capture arms 108 and/or the inflow flange 106 and the RVOT-capture arm 114. The RVOT-capture arm 114 can be constructed from the same material as the central body portion 102 without the use of joints or connections. Suitable methods of construction can include laser cutting from a solid tube or braiding or weaving a wire or wires.

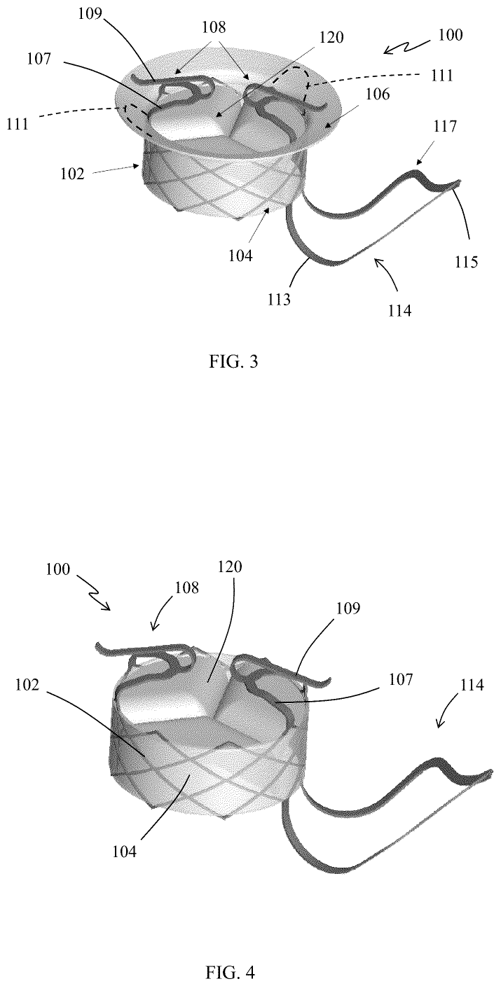

As described herein, the covering layer 104 can limit the flow of blood through the perimeter of the prosthetic heart valve 100 such that blood flow is substantially through the orifice of the prosthetic heart valve 100 (e.g., through the open cylindrical structure defined by the central body portion 102). The covering layer 104 can be attached to the central body portion 102 of the prosthetic heart valve 100 with suture and can be constructed of synthetic materials, such as polyester or polytetrafluoroethylene (PTFE) or biologic materials such as porcine or bovine pericardium. In some arrangements, the prosthetic heart valve 100 can include an inflow flange 106 that extends from the superior perimeter of the prosthetic heart valve to increase sealing against the right atrial surface of the tricuspid valve and prevent or reduce the prosthetic heart valve 100 from migrating inferiorly through the native tricuspid valve due to the interference in diameter of the inflow flange 106 and the native tricuspid valve orifice. In some configurations, the inflow flange 106 can be re-enforced by the central body portion 102, which, for example, can be configured into the shape of the inflow flange 106. In some arrangements, the inflow flange 106 can include a flange-support structure 111 (FIG. 3) disposed at the inflow end of the central body portion 102.

FIG. 2 illustrates a short axis cross section of a heart 2 displaying the mitral valve 18, the pulmonary artery valve 20, the aortic valve 22, and viewing an embodiment of a prosthetic heart valve 100 that is implanted in the tricuspid valve annulus 16 in place of the native tricuspid valve as viewed from the right atrium. As shown in FIG. 2, the prosthetic heart valve 100 can include a plurality of prosthetic valve leaflets 120 that are each secured or fixed to the perimeter of the prosthetic heart valve 100. The radially inward portions of the prosthetic valve leaflets 120 can be free to move or pivot about the edge of the prosthetic valve leaflet 120 that is secured to the perimeter of the prosthetic heart valve 100. In FIG. 2, the prosthetic valve leaflets 120 are shown in a sealed configuration that blocks or prevents blood flow in the direction from the right ventricle to the right atrium (e.g., in a direction out of the illustration). In other words, FIG. 2 illustrates a sealed configuration of the prosthetic leaflets 120, which can occur when the blood pressure in the right ventricle exceeds the blood pressure in the right atrium. In this way, the prosthetic valve leaflets 120 can reduce or prevent FTR. When the blood pressure in the right atrium exceeds the blood pressure in the right ventricle, the prosthetic valve leaflets 120 can pivot or rotate about the fixed perimeter edge in a direction toward the right ventricle and away from the right atrium (e.g. in a direction oriented into the illustration) thereby creating a flow path for blood to flow through the opening formed between the pivoted or rotated prosthetic valve leaflets 120. FIG. 2 shows the prosthetic heart valve 100 can include three prosthetic valve leaflets 120. In some arrangements, the prosthetic heart valve 100 can include only two prosthetic valve leaflets 120 or can include more than three prosthetic valve leaflets 120.

The prosthetic valve leaflets 120 can be constructed from biomaterials, such as porcine or bovine pericardial tissue or synthetic materials and can include two or more prosthetic valve leaflets 120, but preferably three prosthetic valve leaflets 120, attached to the central body portion 102 of the prosthetic heart valve 100 with suture or cord of synthetic construction, such as ultra-high molecular weight polyethylene or polyester. The tricuspid valve repair device is illustrated in FIG. 2 in the right atrium with two tethers 112 spanning the tricuspid valve annulus 16 from the interatrial septum 24 to a pair of locations near the anterior aspect of the tricuspid valve annulus 26. As described herein, the prosthetic heart valve 100 can include a pair of tether capture arms 108 that encircle and capture the tethers 112. A tensioning anchor 122 can secure or anchor the tethers 112 to the interatrial septum 24. In some arrangements, a tensioning anchor 122 can be disposed on the outer surface of the heart 2 to secure the end of the tether 112 that is opposite the interatrial septum 24. The tensioning anchor 122 can be configured as a cushion or as a broad-faced structure that distributes the tension forces of the tethers 112 over a sufficient area of heart tissue (e.g., the interatrial septum wall 24, or the epicardium) to prevent the tethers 112 from cutting into the tissue of the heart 2.

FIG. 3 illustrates a prosthetic heart valve 100 in an expanded or fully-competent-valve configuration. In other words, FIG. 3 shows only the prosthetic heart valve 100 as it would appear implanted into an annulus of a native heart valve while the surrounding tissue of the native heart is not shown in FIG. 3. FIG. 3 illustrates that the tether-capture arms 108 and the RVOT-capture arm 114 can be configured to move between a small-profile configuration (e.g., for delivery through a catheter) and a large-profile configuration (e.g., for forming fully-competent prosthetic heart valve 100 within a native heart valve annulus to reduce or eliminate FTR). For example, the tether-capture arms 108 can include a U-shaped base 107 that is secured at each end to the perimeter of the central body portion 102. The tether-capture arms 108 can further include a top-side extension 109, which in the depicted large-profile configuration is disposed at the midline on the U-shaped base 107 and folds back over the U-shaped base 107. As can be appreciated from FIG. 3, the tether-capture arm 108 can be elongated into a small-profile configuration by extending or pulling the top-side extension 109 and the U-shaped base 108 in the superior direction (e.g., toward the atrium and away from the ventricle) to straighten the tether-capture arm 108 and align the tether-capture arm 108 to be substantially parallel with the longitudinal axis of the central body portion 102 but spaced apart from the longitudinal axis of the central body portion 102 because the tether-capture arm 108 is secured to the perimeter of the central body portion 102. Similarly, the RVOT-capture arm 114 can be brought into a small-profile configuration by extending or pulling a distal end 115 of the RVOT-capture arm 114 in the inferior direction (e.g., toward the ventricle and away from the atrium) to align the RVOT-capture arm 114 with, and spaced apart from, the longitudinal axis of the central body portion 102.

FIG. 3 further illustrates that the RVOT-capture arm 114 can include a spring bend 113 near the end of the RVOT-capture arm 114 that connects to the central body portion 102. The spring bend 113 would straighten (e.g., unbend) when the RVOT-capture arm 114 is moved from the depicted large-profile configuration into the small-profile configuration. In some arrangements, the RVOT-capture arm 114 can further include an intermediate bend 117 disposed between the spring bend 113 and the distal end 115. In some aspects, the intermediate bend 117 can be sized to distribute more evenly onto the RVOT wall the stabilizing forces of the RVOT-capture arm 114. FIG. 3 also shows the prosthetic heart valve 100 can include a flange support 111 that extends or is connected to the central body portion 102. As described for FIG. 1, the flange support 111 can provide additional rigidity to the inflow flange 106 to help the inflow flange 106 resist migration of the prosthetic heart valve 100 (e.g., through interference with the native valve annulus) and can help the inflow flange 106 seal perivalvular blood flow at the perimeter of the prosthetic heart valve 100, for example by biasing the flange 106 in a spring-like fashion to compress against the native valve annulus.

FIG. 4 illustrates a prosthetic heart valve 100 in an expanded or fully-competent-valve configuration, according to some aspects of the present disclosure. The prosthetic heart valve 100 shown in FIG. 4 is similar to the prosthetic heart valve 100 of FIG. 3 except that the prosthetic heart valve 100 shown in FIG. 4 does not include an inflow flange 106. In some aspects, the prosthetic heart valve 100 of FIG. 4 can offer a better option for a patient having a small-diameter native valve annulus. In some arrangements, the inflow flange 106 can be eliminated for a prosthetic heart valve 100 that has a central body 102 sized to exert sufficient radial compression against the native tissue at the perimeter to form a seal against perivalvular blood flow and to inhibit migration of the prosthetic heart valve 100. In other words, in some arrangements, the central body portion 102, the covering layer 104, the tether-capture arms 108, and the RVOT-capture arm 114 can be designed, as disclosed herein, to provide sufficient support to the prosthetic heart valve 100 such that the inflow flange 106 is not required for the sealing of perivalvular blood flow or for the prevention of the migration of the prosthetic heart valve 100.

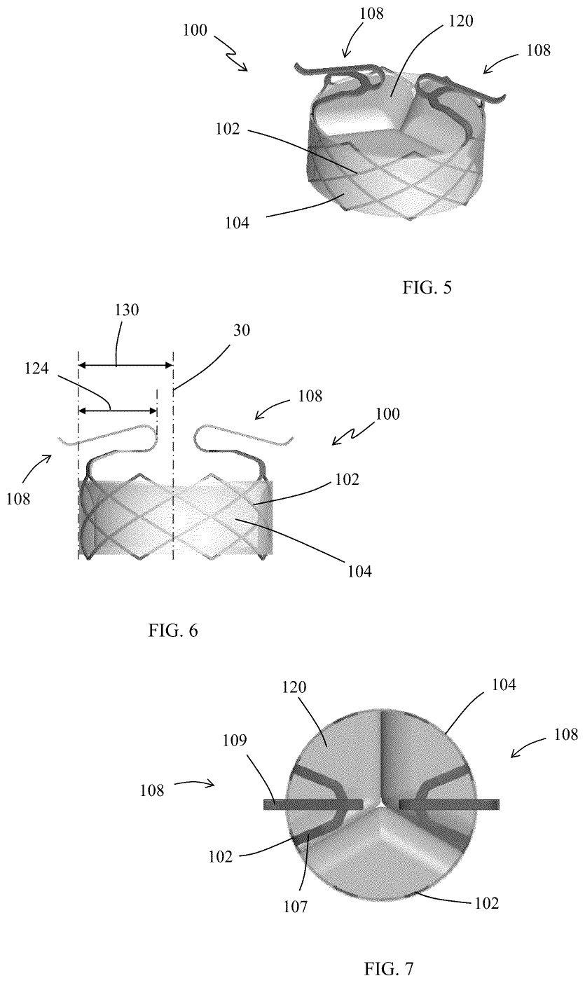

FIG. 5 illustrates a prosthetic heart valve 100 in an expanded or fully-competent-valve configuration, according to some aspects of the present disclosure. The prosthetic heart valve 100 shown in FIG. 5 is similar to the prosthetic heart valve 100 of FIG. 4 except that the prosthetic heart valve 100 shown in FIG. 5 does not include the RVOT-capture arm 114. In some arrangements, the RVOT-capture arm 114 can be eliminated for a prosthetic heart valve 100 that has a central body 102 and tether arm 108 sufficiently sized to prevent migration of the prosthetic heart valve 100.

FIG. 6 illustrates a side view of the prosthetic heart valve 120 shown in FIG. 5. As indicated in FIG. 6, the shape of the tether-capture arm 108 can be characterized by an inward dimension 124 that equals the distance the tether-capture arm 108 extends from the perimeter of the central body portion 102 toward the central axis 30 of the central body portion 102. The central body portion 102 can be characterized by a radius dimension 130. In some aspects, a tether-capture arm ratio can be defined as the ratio between the inward dimension 124 and the radius dimension 130. FIG. 6 shows a prosthetic heart valve 100 having a tether-capture arm ratio of about 0.8. In other words, the tether-capture arm 108 shown in FIG. 6 extends about 80% of the way from the perimeter of the central body portion 102 to the central axis of the central body portion 102. In some arrangements, the prosthetic heart valve 100 can have a tether-capture arm ratio with a value of: 0.2, 0.4, 0.6, 0.7, 0.8, or a value between any of these listed values.

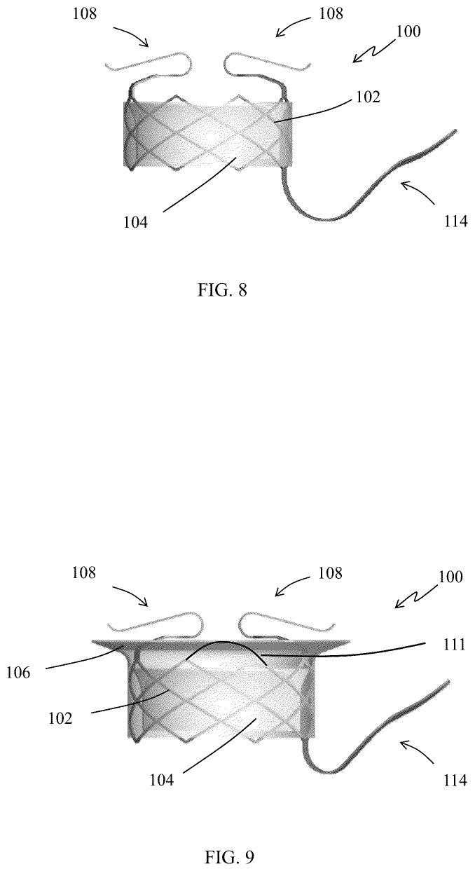

FIG. 7 shows a top view of the prosthetic heart valve 120 shown in FIG. 5. FIG. 7 illustrates that the tether-capture arm 108 can be joined directly to, or can be formed from, the wireframe network of metal struts that form the central body portion 102. For example, FIG. 7 shows that each of the ends of the U-shaped base portion 107 can be connected to, or monolithic with, a metal strut disposed at the superior side of the central body portion 102. FIG. 8 illustrates a side view of the prosthetic heart valve 100 of FIG. 4. FIG. 9 illustrates a side view of the prosthetic heart valve 100 of FIG. 3. FIG. 9 illustrates that the flange support 111 can be joined directly to, or formed from, the wireframe network of metal struts that form the central body portion 102. For example, FIG. 9 shows that each of the ends of the flange support 111 can be connected to, or monolithic with, a metal strut disposed at the inflow (superior) side of the central body portion 102.

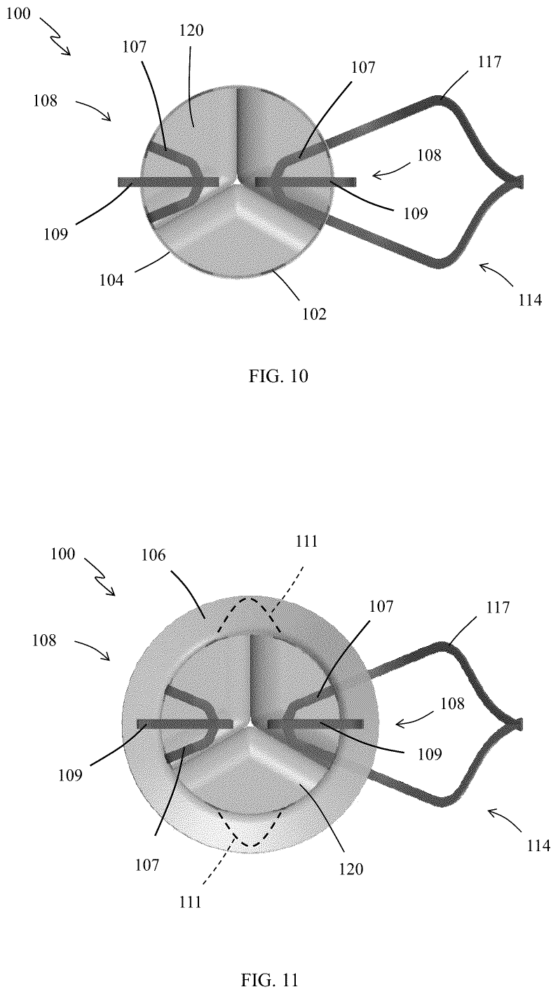

FIG. 10 illustrates a top view of the prosthetic heart valve 100 of FIG. 4. FIG. 11 illustrates a top view of a prosthetic heart valve 100 similar to the prosthetic heart valve 100 of FIG. 3. FIG. 11 shows the flange support structure 111 can be connected to, or monolithic with, the metal struts of the central body portion 102. In some arrangements, the inflow flange 106 can be sufficiently rigid to perform its desired function (e.g., sealing off blood flow at the perimeter of the prosthetic heart valve 100, resisting migration of the prosthetic heart valve 100) without requiring a flange support structure 111 to provide rigidity to the inflow flange 106. FIG. 11 further illustrates that, in some arrangements, the inflow flange 106 can extend radially beyond the tether-capture arm 108.

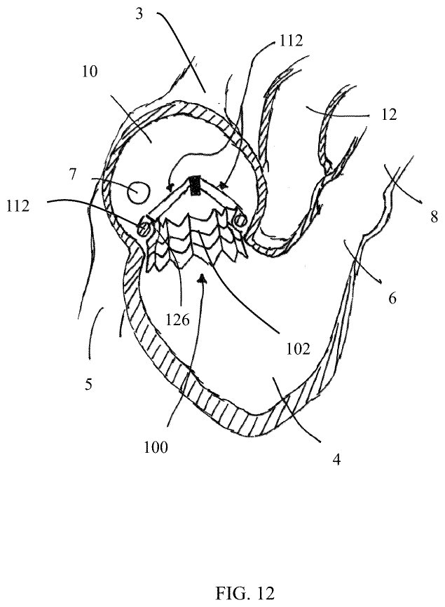

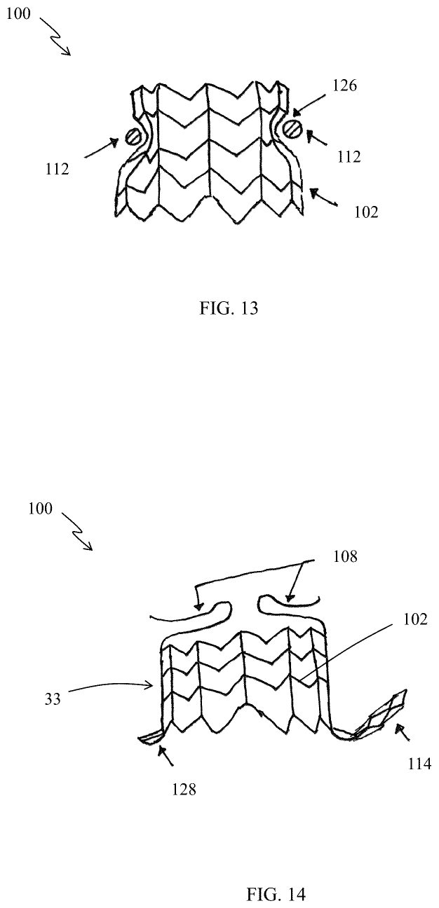

FIG. 12 illustrates an embodiment of a prosthetic tricuspid valve replacement system composed of a tricuspid valve repair system with two tethers 112 spanning the tricuspid valve annulus and a prosthetic heart valve 100 implanted between the two tethers 112. For the sake of clarity, the covering layer 104 is not shown. However, in some arrangements, the prosthetic heart valve 100 can include a covering layer 104, as described herein. FIG. 12 illustrates that the prosthetic heart valve 100 can displace the tethers 112 outward from the original path of the tethers 112 and toward the perimeter of the central body portion 102. Such outward displacement can cause the tethers 112 to shorten relative to the original trajectory of the tethers 112 which in turn causes the force on the tethers 112 to increase and therefore can increase the force pulling the right ventricular free wall and the anterior tricuspid annulus toward the interatrial septum. This can cause the distance between the anterior tricuspid annulus and the interatrial septum to decrease which can squeeze the prosthetic heart valve 100 within the native tricuspid valve annulus and can contribute to migration resistance of the prosthetic heart valve 100. FIG. 13 shows the prosthetic heart valve 100 of FIG. 12 alone and does not show the surrounding heart tissue that would surround the implanted prosthetic heart valve 100.

As can be appreciated from FIGS. 12 and 13, the force exerted by the prosthetic heart valve 100 onto the tethers 112 can also cause the tethers 112 to exert a reactionary squeezing force onto the prosthetic heart valve 100 (e.g., the central body portion 102) which further contributes to fixation of the prosthetic heart valve within the native tricuspid valve annulus. The central body portion 102 of the prosthetic heart valve 100 of FIGS. 12 and 13 can be of similar construction to the central body portion 102 of other arrangements of the prosthetic heart valve 100 described herein and can be compliant (e.g., through selection of the material properties and strut thicknesses of the wireframe of the central body 102) such that the tethers 112 cause the central body portion 102 to indent radially at the locations of contact with the tethers 112. In some aspects, the indentation 126 of the central body portion 102 can be within the range from 1 mm to 10 mm. These indentations 126 can increase the gripping effect between the tethers 112 and the prosthetic heart valve 100 and, therefore can increase the resistance of the prosthetic heart valve 100 to migration. Alternatively, a covering material such as described herein (e.g., covering layer 104) can be disposed over the central body portion 102 and can be compliant such that the covering material is indented from the squeezing force exerted by the tethers 112, with the same benefits as the central body portion 102 being compliant. For the sake of clarity, the covering material is not shown in FIG. 13, but if such a covering material were applied to the prosthetic heart valve 100 it would be disposed over the central body portion 102 and would also fill the gap that appears in FIG. 13 between the suture 112 and the central body portion 102 at the indentation 126.

FIG. 14 illustrates a side profile of an embodiment of a prosthetic heart valve 100 with the features and benefits described herein and additionally configured with a posterior support arm 128 radiating outward from the outflow side of the central body portion 102. As depicted in FIG. 14, the posterior support arm 128 can be disposed approximately circumferentially opposite from a RVOT-capture arm 114. As described with regard to the RVOT-capture arm 114, the posterior support arm 128 can be flexible such that it can be elongated and compressed into a delivery catheter and can be in the configuration of a small hook or shelf. When hemodynamic forces are exerted on the prosthetic heart valve 100, a moment could be created about the RVOT-capture arm 114 which may cause the prosthetic heart valve 100 to begin rotating with the posterior side 33 of the prosthetic heart valve 100 rotating superiorly. The posterior support arm 128 can counteract this moment and prevent rotation by bracing the prosthetic heart valve 100 against the right ventricular surface of the posterior tricuspid valve annulus. The posterior support arm 128 can be constructed using the same materials and methods as described for the RVOT-capture arm 114.

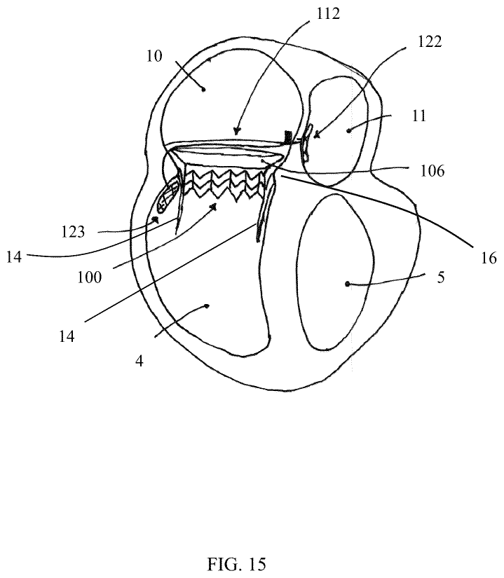

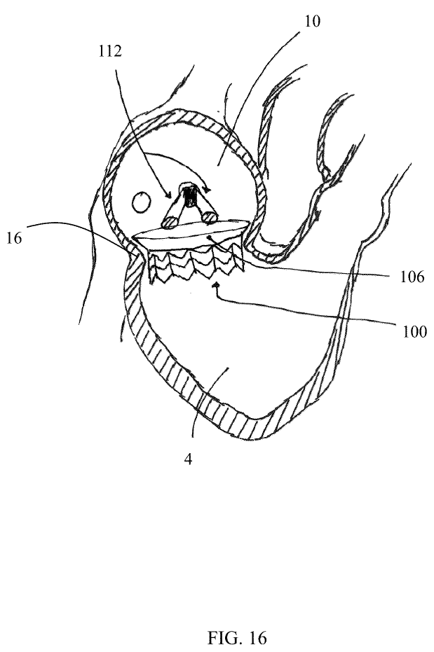

FIGS. 15 and 16 illustrate an embodiment of a tricuspid valve replacement system comprised of a tricuspid valve repair system with one or more tethers 112 spanning the native tricuspid valve annulus 16 and a prosthetic heart valve 100 implanted within the native tricuspid valve annulus 16 with an inflow flange 106 on the inflow side of the prosthetic heart valve 100. The inflow flange 106 can be configured to be implanted between the one or more tethers 112 and the right atrial surface of the native tricuspid valve annulus. The inflow flange 106 can be configured to have a diameter that is larger than the native tricuspid valve annulus 16. The prosthetic heart valve 100 can thus be trapped between the tethers 112 and the native annulus 16 and thereby inhibit migration of the prosthetic heart valve 100 superiorly due to the tethers 112 or inferiorly due to the native tricuspid valve annulus 16. The prosthetic heart valve 100 can be constructed using similar materials and methods as those described for other embodiments disclosed herein.

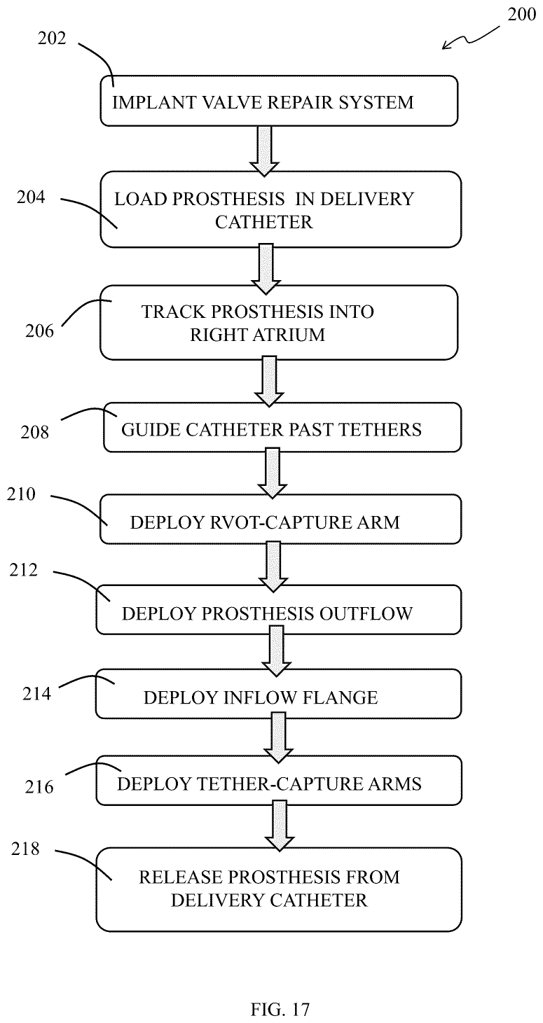

FIG. 17 illustrates a method of tricuspid valve replacement 200 that can be used to implant a prosthetic replacement tricuspid valve (e.g., a prosthetic heart valve 100) into a native heart valve annulus of a patient. The method 200 can include a repair-system-implantation step 202 in which a tricuspid valve repair system is implanted into the patient, as described in International PCT Patent Application No. PCT/US2023/020277. The method 200 can further include a prosthetic-loading step 204 in which the prosthetic heart valve 100 is placed into a low-profile configuration and loaded into a catheter suitable for delivery into the right atrium of a heart. The method 200 can further include a tracking step 206 in which the delivery catheter containing the loaded heart valve prosthesis 100 can be used to track the prosthetic heart valve 100 through a patient's femoral vein, inferior vena cava, and into the right atrium. The method 200 can further include a deflection step 208 in which the delivery catheter can be deflected toward the tricuspid annulus and can be guided through or past the tethers 112 that have been installed with the tricuspid valve repair system. In the case where there are two tethers, the delivery catheter can be guided between the two tethers 112 in the deflection step 208. The method 200 can include deploying the prosthetic tricuspid valve into a right atrium of the heart, and the deployment steps can depend on the characteristics of the prostheses that is being implanted. For example, if the prostheses is a prosthetic heart valve 100 equipped with an RVOT-capture arm 114, the method 200 can further include a RVOT-capture-arm-deployment step 210 in which the RVOT-capture arm 114 is deployed. The method 200 can further include an outflow-deployment step 212 in which the inferior portion of the central body portion 102 can be deployed into the native tricuspid valve orifice. The method 200 can further include an inflow-flange-deployment step 214, in which, if the prosthetic heart valve 100 is equipped with an inflow flange 106, the inflow flange 106 can be deployed on the inflow side between the tethers 112 and the right atrial surface of the native tricuspid valve annulus 16. The method 200 can further include a tether-capture-arm-deployment step 216, in which, if the prosthetic heart valve 100 is equipped with tether capture arms 108, the catheter delivery catheter can be pulled back to implant the tether capture arms 108 such that the tether-capture arms 108 may capture the tethers 112. In some arrangements, the method 200 can include deploying the prosthetic heart valve 100 between at least two tethers 112 such that the central body 102 is squeezed between the at least two tethers 112 (see, e.g., FIG. 13). The method 200 can further include a prosthesis-release step 218, in which the prosthetic heart valve 100 can be released from the delivery catheter, and the delivery catheter can be removed from the patient.

While certain embodiments of the inventions have been described, these embodiments have been presented by way of example only and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

The scope of the present disclosure is not intended to be limited by the specific disclosures of preferred embodiments in this section, or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A prosthetic heart valve comprising:
a central body portion comprising a superior end and an inferior end, the central body portion defining a tube-like structure that connects the superior end to the inferior end; and
a tether-capture arm disposed on the superior end and comprising an attached end that connects to the central body, the tether-capture arm further comprising a free end, the tether-capture arm forming a U-shaped tether-capture notch by extending from the attached end in a radially inward direction and further extending in a radially outward direction to terminate at the free end.

2. The prosthetic heart valve of claim 1, further comprising a covering layer disposed on the central body portion, the covering layer configured to inhibit a fluid flow in a radial direction of the tube-like structure.

3. The prosthetic heart valve of claim 1, wherein the central body portion is configured to move between a first state and a second state, a diameter of the central body within a range of 4 mm to 13 mm in the first state, the diameter of the central body within the range of 25 mm to 60 mm in the second state.

4. The prosthetic heart valve of claim 1, further comprising two or more prosthetic valve leaflets disposed at the superior end, the two or more prosthetic valve leaflets configured to allow a fluid flow through the hollow structure in a direction from the superior end to the inferior end and further configured to inhibit the fluid flow through the hollow structure in a direction from the inferior end to the superior end.

5. The prosthetic heart valve of claim 1, wherein the tether-capture notch is disposed superior to the attached end.

6. The prosthetic heart valve of claim 1, wherein the free end of the tether-capture arm is disposed superior to the attached end.

7. The prosthetic heart valve of claim 1, wherein the free end of the tether-capture arm is disposed radially outward of the attached end.

8. The prosthetic heart valve of claim 1, further comprising an inflow flange disposed at a perimeter of the superior end and extending radially beyond the superior end of the central body portion such that an outer dimension of the inflow flange is larger than an outer dimension of the superior end of the central body portion.

9. The prosthetic heart valve of claim 1, further comprising a tether seated within the tether-capture notch.

10. The prosthetic heart valve of claim 1, further comprising a right ventricular outflow tract (RVOT) capture arm disposed at the inferior end of the central body, the RVOT-capture arm comprising an attached end that connects to the central body, the RVOT-capture arm further comprising a free end disposed superior to the attached end.

11. The prosthetic heart valve of claim 10, further comprising a posterior support arm disposed at the inferior end and circumferentially spaced apart from the attached end of the RVOT-capture arm.

12. A prosthetic heart valve comprising:
a central body portion comprising a superior end and an inferior end, the central body portion defining a hollow structure that connects the superior end to the inferior end; and
a tether disposed longitudinally between the superior end and the inferior end, the tether configured to compress the hollow structure in a radial direction thereby forming a tether-capture notch on an outer surface of the hollow structure.

13. The prosthetic heart valve of claim 12, further comprising a covering layer disposed on the central body portion and configured to inhibit a fluid flow in a radial direction of the hollow structure.

14. The prosthetic heart valve of claim 12, wherein a depth dimension of the tether-capture notch is within a range of 1 mm to 10 mm.

15. The prosthetic heart valve of claim 12, wherein the central body portion is configured to move between a first state and a second state, a diameter of the central body within a range of 4 mm to 13 mm in the first state, the diameter of the central body within the range of 25 mm to 60 mm in the second state.

16. The prosthetic heart valve of claim 12 further comprising two or more prosthetic valve leaflets disposed at the superior end, the two or more prosthetic valve leaflets configured to allow a fluid flow through the hollow structure in a direction from the superior end to the inferior end and further configured to inhibit the fluid flow through the hollow structure in a direction from the inferior end to the superior end.

17. A prosthetic heart valve comprising:
a central body portion comprising a superior end and an inferior end, the central body portion defining an open cylindrical structure that connects the superior end to the inferior end;
an inflow flange disposed at a perimeter of the superior end and extending radially beyond the perimeter of the superior end; and
a tether disposed superior to the inflow flange and configured to inhibit the inflow flange from moving in a superior direction.

18. The prosthetic heart valve of claim 17, further comprising a covering layer disposed on the central body portion and configured to inhibit a fluid entering or exiting the open cylindrical structure in a radial direction of the open cylindrical structure.

19. The prosthetic heart valve of claim 17, wherein the central body portion is configured to move between a first state and a second state, a diameter of the central body within a range of 4 mm to 13 mm in the first state, the diameter of the central body within the range of 25 mm to 60 mm in the second state.

20. The prosthetic heart valve of claim 17 further comprising a flange support structure connected to the central body portion and extending into the inflow flange.

21. A method of tricuspid valve replacement, the method comprising:
implanting a tricuspid valve repair system into a heart, the tricuspid valve repair system having at least one tether that spans a native tricuspid valve annulus of the heart;
loading a prosthetic replacement tricuspid valve into a delivery catheter;
tracking the prosthetic replacement tricuspid valve into a right atrium of the heart; and
deploying the prosthetic tricuspid valve into the native tricuspid valve annulus.

22. The method of claim 21, further comprising: deploying an inflow flange of the prosthetic replacement tricuspid valve between the at least one tether and the native tricuspid valve annulus.

23. The method of claim 21, further comprising: deploying at least one tether-capture arm of the prosthetic replacement tricuspid valve to capture at least one tether of the tricuspid valve repair system.

24. The method of claim 21, wherein the tricuspid valve repair system includes at least two tethers that span the native tricuspid valve annulus, the method further comprising: deploying the prosthetic replacement tricuspid valve between the at least two tethers such that a central body portion of the prosthetic replacement tricuspid valve is squeezed between the at least two tethers.
